(19) United States
(12) Patent Application Publication
(10) Pub. No.: US 2023/0393116 A1
(43) Pub. Date: Dec. 7, 2023

(54) BIOMARKER FOR BLOOD ANOMALY

(71) Applicant: INDIAN INSTITUTE OF SCIENCE, Bangalore (IN)

(72) Inventors: Siva UMAPATHY, Bangalore (IN); Dipankar NANDI, Bangalore (IN); Ravindran BALACHANDRAN, Bangalore (IN); Taru VERMA, Bangalore (IN)

(21) Appl. No.: 18/032,832

(22) PCT Filed: Dec. 16, 2021
(86) PCT No.: PCT/IB2021/061834

§ 371 Date: Apr. 20, 2023

(30) Foreign Application Priority Data
Oct. 21, 2020 (IN) 202041045902

(51) Int. Cl. G01N 33/483; G01N 21/65

(52) U.S. Cl. CPC G01N 33/483; G01N 21/65

(57) ABSTRACT

The invention provides a method of obtaining a biomarker for blood anomaly using Raman scattering. The method includes the first step of capturing at least two unique Raman signatures with respect to the biomarker. The Raman signatures are obtained at regular interval. Second step is comparing the obtained unique Raman signatures to detect the change in the signature. Change in the Raman signature provides the indication for blood anomaly.

### FIELD OF INVENTION

The invention generally relates to the field of Raman scattering and particularly to a method of obtaining a biomarker for blood anomaly using Raman scattering.

### BACKGROUND OF THE INVENTION

Blood anomaly in a person can lead to life-threatening conditions. Blood anomaly includes but is not limited to sepsis and septic shock. Sepsis results from an individual's response to infection or trauma. The body normally releases chemicals into the bloodstream to fight an infection. Sepsis occurs when the body's response to these chemicals is out of balance, triggering changes that can damage multiple organ systems. While sepsis can occur in anyone, this condition is often observed in immunocompromised individuals, children and the elderly population post infection. If sepsis progresses to septic shock, blood pressure drops dramatically. This may lead to death. Sepsis is difficult to diagnose and has become the primary cause of mortality in ICUs.

The major factors responsible for increase in the cases of sepsis includes but are not limited to rise in the number of organ transplants and other surgical procedures that require suppressing the patient's immune system; increase in the number of elderly people in the population; and rampant use of antibiotics to treat infections, resulting in a higher incidence of multi-drug resistant bacterial strains.

The importance of early diagnosis of sepsis cannot be overlooked. Rapid and reliable diagnostic methods and technologies for sepsis can prove to be beneficial especially if they can exist as point-of-care tests or devices. Once sepsis is diagnosed, appropriate antibiotic therapy is often life-saving.

Various approaches involved in diagnosis of sepsis include but are not limited to blood culture analysis, nucleic acid-based tests, fluorescence in situ hybridization method, and DNA microarrays. Blood culture is considered to be the standard for blood stream infections diagnosis. This technique allows accurate identification of the pathogens and subsequent anti-microbial therapies. However, this technique has major disadvantages including requirement of large volumes of blood samples and longer time period for obtaining results. Nucleic acid-based tests have been introduced where in detection of the causative microorganism is relatively faster. Although this assay is easy to perform, but obtains many false positive results. Fluorescence in situ hybridization method leads to reporting delays, as pathogens are required to grow on the medium. DNA microarrays have also been used for diagnosis of sepsis. This system is particularly beneficial as along with identification of the causative pathogen, susceptibility towards antibiotics and virulence genes can also be evaluated. The drawback of this method is that discrimination between highly similar target sequences becomes difficult. Therefore, there is a need for a diagnostic measure that is minimally-invasive, label-free, requires extremely low sample amounts and preparation as well as yields results rapidly without any false negative results.

### SUMMARY OF THE INVENTION

One aspect of the invention provides a method of obtaining a biomarker for blood anomaly using Raman scattering. The method includes the first step of capturing at least two unique Raman signatures with respect to the biomarker. The Raman signatures are obtained at regular interval. Second step is comparing the obtained unique Raman signatures to detect the change in the signature. Change in the Raman signature provides indication for blood anomaly.

### BRIEF DESCRIPTION OF DRAWINGS

So that the manner in which the recited features of the invention can be understood in detail, some of the embodiments are illustrated in the appended drawings. It is to be noted, however, that the appended drawings illustrate only typical embodiments of this invention and are therefore not to be considered limiting of its scope, for the invention may admit to other equally effective embodiments.

FIG. 1 shows average Raman spectra of plasma from healthy controls, sepsis and shock patients, according to an example of the invention.

FIG. 2a shows Raman signature corresponding to Tyrosine Fermi doublet distinguishing sepsis and shock from healthy controls, according to an example of the invention.

FIG. 2b shows Raman signature corresponding to C—C skeletal stretching from amino acids distinguishing sepsis and shock from healthy controls, according to an example of the invention.

FIG. 2c shows Raman signature corresponding to C—C skeletal stretching in proteins distinguishing sepsis and shock from healthy controls shows, according to an example of the invention.

FIG. 2d shows Raman signature corresponding to ratio of Alpha helix to random coils distinguishing sepsis and shock from healthy controls shows, according to an example of the invention.

FIG. 3 shows Resonant Raman spectra of sepsis and shock patients using 514 nm laser excitation, according to an example of the invention.

FIG. 4a shows Raman signature at 1156 cm⁻¹ corresponding to Carotenoids differentiating sepsis from shock, according to an example of the invention.

FIG. 4b shows Raman signature at 1525 cm⁻¹ corresponding to Carotenoids differentiating sepsis from shock, according to an example of the invention.

### DETAILED DESCRIPTION OF THE INVENTION

Various embodiments of the invention provide a method for obtaining a biomarker for blood anomaly using Raman scattering.

The method includes the first step of capturing at least two unique Raman signatures with respect to the biomarker. The Raman signatures are obtained at regular interval. Second step is comparing the obtained unique Raman signatures to detect the change in the signature. Change in the Raman signature provides the indication for blood anomaly. The method described herein, briefly shall be discussed in detail, herein below.

The method for obtaining biomarker for blood anomaly is based on Raman scattering. Raman spectroscopy measures the spectra of microscopic samples. The technique captures molecular bond specific vibrations originating from the biochemical constituents. Biochemical constituents include but are not limited to DNA, RNA, proteins, lipids, carbohydrates. Different forms of Raman spectroscopy include Resonant Raman spectroscopy and Non-resonant Raman spectroscopy.

First step of obtaining biomarker for blood anomaly using Raman scattering includes capturing at least two unique Raman signatures with respect to the biomarker. Second step is comparing the obtained unique Raman signatures to detect the change in the Raman signature. The Raman signatures are obtained at regular interval. Change in the Raman signature provides the indication for blood anomaly. The blood anomaly includes but is not limited to sepsis and septic shock.

The biomarker for blood anomaly includes but is not limited blood anomaly related protein denaturation as well as differences in the antioxidant molecules. The biomarker is known to show changes on the onset of blood anomaly.

In one embodiment of the invention, biomarker includes Tyrosine Fermi doublet, C—C skeletal stretching in protein, ratio of Alpha helix to random coils in protein, carotenoids.

Plasma samples are drop-cast onto a suitable Raman substrate including but not limited to Magnesium fluoride coverslip. Raman signatures are collected in transmission mode.

According to an embodiment of the invention, Raman signatures are obtained through non-resonant Raman scattering. The laser wavelength chosen is in the near IR region in the range from 500 nm to 1024 nm.

According to another embodiment of the invention, Raman signatures are obtained through resonant Raman scattering. The laser wavelength chosen is in the range from 300 nm to 600 nm.

All spectral recordings are performed using 50× objective. Raman signatures are collected using Wire 4.1 software. Data processing is performed using MATLAB and Origin 2016 software. Multivariate analysis is performed using CAMO Unscrambler software.

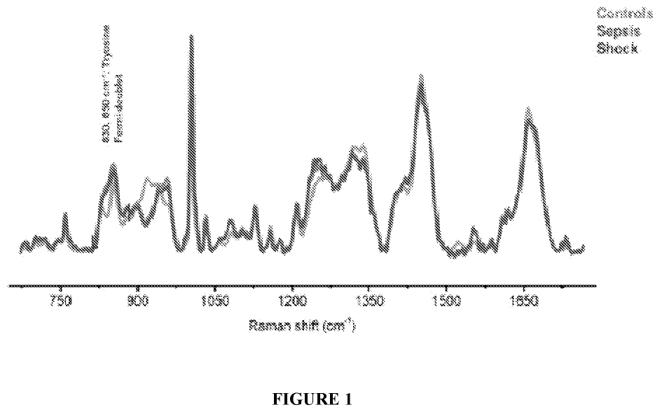

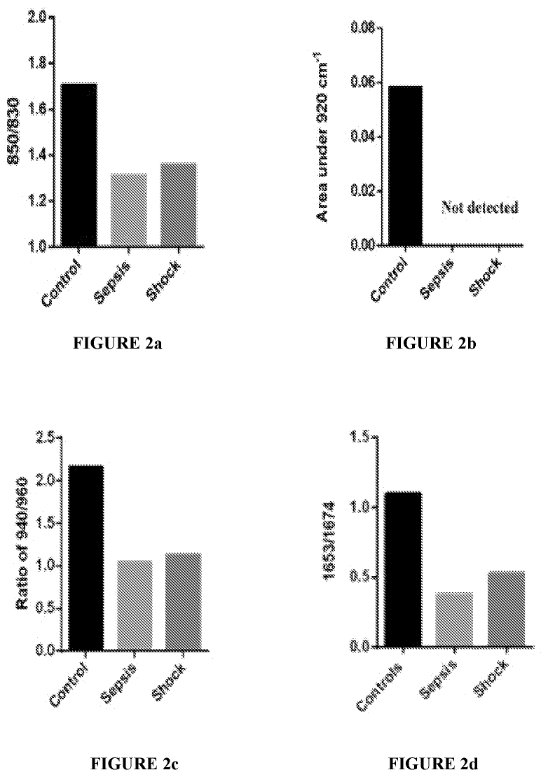

In one embodiment of the invention, Raman signatures are pre-processed using a series of steps so that peaks resolve better. The pre-processing steps include Derivatization and smoothing. The captured Raman signatures are subjected to multivariate curve resolution to identify major chemical constituents. Many of the resolved peaks have predefined assignments to specific chemical bonds. FIG. 1 show the average Raman spectra of plasma from healthy controls, sepsis and shock patients recorded under non-resonant conditions using 785 nm laser excitation, according to an example of the invention. Tyrosine Fermi doublet having Raman bands at 830 and 854 cm⁻¹ as shown in FIG. 2a depicts protein denaturation and appears to be convoluted in the sepsis and septic shock category when compared to the controls. The difference is very prominent and easily identifiable. FIG. 2b shows absence of the Raman band at 920 cm⁻¹ corresponding to C—C skeletal stretching from amino acids, in the sepsis and shock samples. FIG. 2b provides characteristic difference identified using Raman spectroscopy.

FIG. 2c shows decrease in intensity ratio of two Raman bands at 940 and 960 cm⁻¹, both of these have contributions from C—C skeletal stretching in proteins.

Protein structural differences can also be identified using Raman spectroscopy using the bands at 1252 cm⁻¹ (Amide III) as well as 1660 and 1678 cm⁻¹ (Amide I). An increase in the band at 1252 and 1678 cm⁻¹ is observed for sepsis and shock while a reduction is seen in the band at 1660 cm⁻¹ which is a marker band for alpha helix. The ratio of 1660/1678 cm⁻¹ corresponding to Alpha helix to random coils, shows a reduction in sepsis and shock compared to controls. This indicates extensive protein denaturation occurring as a result of sepsis, as shown in FIG. 2d.

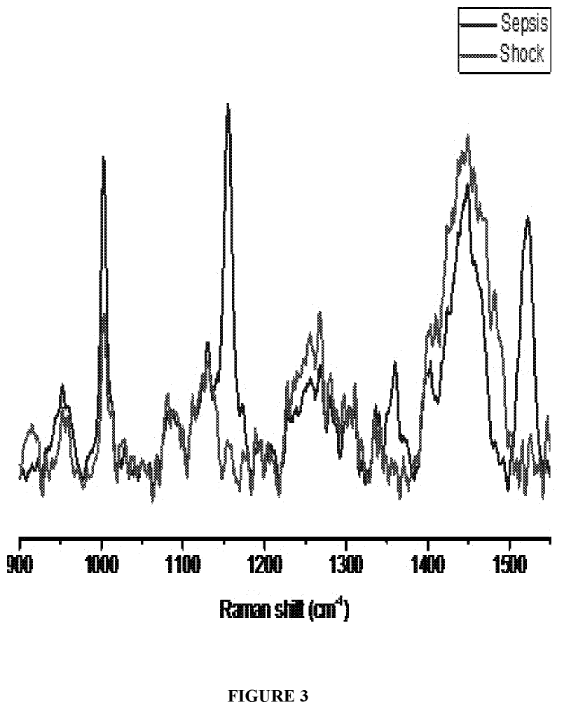

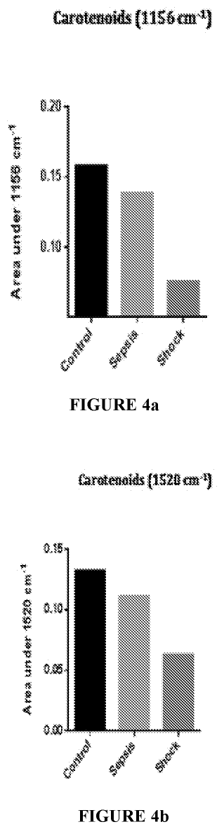

In one example of the invention, Resonance Raman spectroscopy is used to differentiate different grades of sepsis on the basis of carotenoids as the biomarker. FIG. 3 shows Resonant Raman spectra of sepsis and shock patients using 514 nm laser excitation. Shock samples have significantly reduced carotenoid levels when compared to the sepsis samples. The intensity of all the carotenoids Raman bands at 1156 and 1525 cm⁻¹ reduces in the shock plasma sample compared to the sepsis category. FIG. 4 a and FIG. 4b shows Carotenoids serve as a biomarker for differentiating sepsis from shock 1156 cm⁻¹ and 1525 cm⁻¹ , respectively.

The foregoing description of the invention has been set merely to illustrate the invention and is not intended to be limiting. Since modifications of the disclosed embodiments incorporating the spirit and substance of the invention may occur to person skilled in the art, the invention should be construed to include everything within the scope of the appended claims and equivalents thereof.

We claim:

1) A method of obtaining a biomarker for blood anomaly using Raman scattering, the method comprising:
capturing at least two unique Raman signatures with respect to the biomarker, wherein the signatures are obtained at regular interval; and
comparing the obtained unique signatures to detect the change in the signature.
Wherein, the change in the signature provides the indication for blood anomaly.

2) The method as claimed in claim 1, wherein the blood anomaly is sepsis or septic shock.

3) The method according to claim 1, wherein the Raman scattering is Resonant Raman scattering or Non-resonant Raman scattering.

4) The method as claimed in claim 1, wherein the wavelength of the electromagnetic radiation for Resonant Raman scattering is in the range of 300 nm to 600 nm.

5) The method as claimed in claim 1, wherein the wavelength of the electromagnetic radiation for Non-resonant Raman scattering is in the range of 500 nm to 1024 nm.

6) The method as claimed in claim 1, wherein the biomarker is selected from Tyrosine Fermi doublet, C—C skeletal stretching in protein, ratio of Alpha helix to random coils in protein, carotenoids.
